(19) United States
(12) Patent Application Publication
(10) Pub. No.: US 2003/0198603 A1
(43) Pub. Date: Oct. 23, 2003

(54) Hair lacquer based on branched sulphonic polyesters with a high water content

(76) Inventors: Isabelle Rollat-Corvol, Paris (FR); Christine Dupuis, Paris (FR); Katarina Benabdillah, Clichy (FR); Christiane Condamine, Eragny S/Oise (FR)

Correspondence Address: NIXON & VANDERHYE, PC, 1100 N GLEBE ROAD, 8TH FLOOR, ARLINGTON, VA, 22201-4714, US

(21) Appl. No.: 10/320,707

(22) Filed: Dec. 17, 2002

(30) Foreign Application Priority Data
Dec. 17, 2001 (FR) 0116318

(51) Int. Cl. A61K 9/00; A61K 7/06; A61K 7/11

(52) U.S. Cl. 424/47; 424/70.11

(57) ABSTRACT

The invention relates to a styling composition packaged in an aerosol device, comprising

from 45% to 65% by weight of a liquid phase containing, in dissolved or finely dispersed form in a cosmetically acceptable aqueous or aqueous-alcoholic liquid medium, at least one branched sulphonic polyester, and

from 35% to 55% of dimethyl ether as propellant,

the total water content of the liquid phase being between 65% and 99% by weight, and also to a styling process using such a composition.

The present invention relates to styling compositions packaged in an aerosol device, comprising a branched sulphonic polyester and a large proportion of water, and also to a styling process using such a composition.

The use of branched sulphonic polyesters in hair styling and fixing compositions is known and described, for example, in documents EP 0 966 946, WO 98/38969 and WO 99/63955.

However, the Applicant has found that, although the use of these branched sulphonic polyesters in hair lacquers containing large proportions of alcohol gives excellent styling properties, they do not make it possible to obtain sufficient lacquering power.

Moreover, branched sulphonic polyesters applied in the form of lacquers with a high alcohol content give the hair a dry feel, after removal by brushing. This undesirable phenomenon is particularly noticeable for dyed hair.

The Applicant has found that increasing the overall water content of hair lacquers not only improves the lacquering power of the compositions obtained, but also gives the hair, after brushing, a smoother and more cosmetic feel than that obtained with the known lacquers with a high alcohol content.

One subject of the invention is, consequently, a styling composition packaged in an aerosol device, comprising

from 45% to 65% by weight of a liquid phase containing, in dissolved or finely dispersed form in a cosmetically acceptable aqueous or aqueous-alcoholic liquid medium, at least one branched sulphonic polyester, and

from 35% to 55% by weight of dimethyl ether as propellant,

the total water content of the liquid phase being between 65% and 99% by weight.

A subject of the invention is also a process for styling by spraying such a styling composition, packaged in an aerosol device, onto the hair.

As indicated above, the liquid phase of the styling composition of the present invention contains an aqueous or aqueous-alcoholic medium, namely a mixture of water and ethanol.

When it contains ethanol, the weight ratio of the ethanol present in the liquid phase to the dimethyl ether (propellant) is preferably less than or equal to 1.

In one preferred embodiment, the ethanol content of the liquid phase is less than or equal to 35% by weight, preferably less than or equal to 25% by weight and in particular less than or equal to 15% by weight.

The branched sulphonic polyesters used in the compositions of the present invention are known in the art. Their structure and synthesis are described in documents WO 95/18191, WO 97/08261 and WO 97/20899.

Branched sulphonic polyesters that are particularly preferred are those obtained by polycondensation

(a) of at least one dicarboxylic acid not bearing a sulphonic function,

(b) of at least one diol or a mixture of a diol and a diamine,

(c) of at least one monomer comprising two reactive functions, which may be identical or different, chosen from hydroxyl, amino and carboxyl groups, and also bearing at least one sulphonic function, and

(d) of at least one monomer comprising at least three reactive functions, which may be identical or different, chosen from hydroxyl, amino and carboxyl groups.

The dicarboxylic acids forming the units (a) may be aliphatic dicarboxylic acids, alicyclic dicarboxylic acids and aromatic dicarboxylic acids, mixtures of such acids.

Examples that may be mentioned include 1,4-cyclohexanedioic acid, succinic acid, glutaric acid, adipic acid, azelaic acid, sebacic acid, fumaric acid, maleic acid, 1,3-cyclohexanedioic acid, phthalic acid, terephthalic acid and isophthalic acid, and a mixture of such acids.

The diols forming the units (b) are chosen, for example, from alkanediols and polyalkylenediols, and examples that may be mentioned include ethylene glycol, propylene glycol, diethylene glycol, triethylene glycol and polypropylene glycol.

The diamines capable of forming part of the units (b) are preferably chosen from alkanediamines and poly(oxyalkylene)diamines.

The term “sulphonic function” of the units (c) includes both the sulphonic acid function (—SO₃H) and the corresponding salified functions obtained by neutralization of the sulphonic acid function with a base, for example with an alkali metal hydroxide.

The sulphonic functions are preferably in the form neutralized with an organic or mineral base.

The units (c) are derived, for example, from dicarboxylic acids, dicarboxylic acid esters, glycols and hydroxy acids, all bearing at least one sulphonic group, in acid and/or neutralized form, preferably in neutralized form.

The units (c) bearing at least one sulphonic function preferably represent from 2 mol% to 15 mol% of the total amount of monomers.

The units (d) derived from multifunctional monomers are preferably present in an amount of between 0.1 mol% and 40 mol% relative to the total amount of monomers.

The multifunctional monomers forming the units (d) are chosen, for example, from trimethylolethane, trimethylolpropane, glycerol, pentaerythritol, sorbitol, trimellitic anhydride, erythritol, threitol, dipentaerythritol, pyromellitic dianhydride and dimethylpropionic acid.

The branched sulphonic polyesters may comprise, in addition to the four types of units (a) to (d) described above, units (e) derived from monomers comprising two different reactive functions, chosen, for example, from hydroxy carboxylic acids and amino carboxylic acids, or a mixture thereof.

These units (e) may represent up to 40 mol% of the total amount of monomers (a), (b), (c), (d) and (e).

Needless to say, the branched sulphonic polyesters used in the present invention are preferably obtained from a mixture of monomers in which the number of equivalents of carboxylic acid functions is substantially equal to the number of equivalents of hydroxyl functions and of amino functions, which may be present.

The branched sulphonic polyesters therefore have a high molecular weight. The number-average molecular weight (NAMW, determined by size exclusion chromatography) of the branched sulphonic polyesters used in the present invention is preferably >5000 g/mol. Their polydispersity is >3, preferably about 4.

The branched sulphonic polyesters used in the styling compositions of the present invention are known and sold, for example, by the company Eastman. A preferred commercial product that may be mentioned is the product sold under the name AQ 1350® by the company Eastman.

The styling compositions of the present invention preferably have a water content in the liquid phase of between 70% and 98% by weight and preferably between 75% and 96% by weight.

The liquid phase may contain hydroxylated hydrocarbon-based compounds other than ethanol, such as, for example, glycerol and isopropanol.

The liquid phase, also known as the “fluid”, of the styling compositions of the present invention must have a minimum concentration of branched sulphonic polyester allowing the deposition of a sufficient amount of polymer for fixing the hair.

In general, it has been found that a branched sulphonic polyester concentration of between 0.2% and 15% relative to the total weight of the liquid phase of the styling composition is entirely suitable for the invention. A concentration range extending from 0.5% to 10% of the weight of the liquid phase is preferred in particular.

As indicated above, the propellant, i.e. the dimethyl ether, represents from 35% to 55% of the total weight of the aerosol styling composition of the present invention.

The styling compositions of the present invention may contain—in addition to one or more branched polyesters—one or more other known anionic, nonionic, amphoteric or cationic film-forming fixing polymers in dissolved or dispersed form (latex).

The styling compositions of the present invention may also contain one or more cosmetic or formulation additives usually used in the cosmetics field. Examples of such additives that may be mentioned include UV screening agents, fragrances, preserving agents, pigments and colorants, solubilizing agents, antifoams, vitamins, conditioners such as dispersed or microdispersed soluble silicones, organic or mineral, synthetic or non-synthetic particles, and surfactants.

The styling compositions of the present invention are preferably hair lacquers.

### EXAMPLE

A hair lacquer A with a high water content, according to the invention, and a comparative hair lacquer B with a low water content, having the compositions below, are prepared:

| | Lacquer A | |
|---|---|---|
| | (according to the | Lacquer B |
| Ingredients | invention) | (comparative) |
| Branched | 3.5% by weight of | 3.5% by weight of |
| sulphonic | active material | active material |
| polyester | (a.m.) (i.e. 5.4% | (i.e. 5.4% by |
| AQ 1350 | by weight of a.m. | weight relative to |
| (Eastman) | relative to the | the liquid phase) |
| | liquid phase) | |
| Water | 41.5% by weight | 26.5% by weight |
| | (i.e. 63.8% | (i.e. 40.8% |
| | relative to the | relative to the |
| | liquid phase) | liquid phase) |
| Ethanol | 20% by weight | 35% by weight (i.e. |
| | (i.e. 30.8% | 53.8% relative to |
| | relative to the | the liquid phase) |
| | liquid phase) | |
| Dimethyl ether | 35% by weight | 35% by weight |

The two lacquers are sprayed onto models. Each composition is sprayed in comparison on one half of the head. The results are assessed by sensory evaluation by a trained expert.

The lacquering power is greater for lacquer A according to the invention than for the comparative lacquer B. After brushing, the hair is smoother and less dry for lacquer A according to the invention.

1. Styling composition packaged in an aerosol device, comprising
from 45% to 65% by weight of a liquid phase containing, in dissolved or finely dispersed form in a cosmetically acceptable aqueous or aqueous-alcoholic liquid medium, at least one branched sulphonic polyester, and
from 35% to 55% by weight of dimethyl ether as propellant,
the total water content of the liquid phase being between 65% and 99% by weight.

2. Styling composition according to claim 1, characterized in that it contains ethanol and in that the ethanol/dimethyl ether weight ratio is less than or equal to 1.

3. Styling composition according to claim 2, characterized in that the liquid phase has an ethanol content of less than or equal to 35% by weight, preferably less than or equal to 25% by weight and in particular less than or equal to 15% by weight.

4. Styling composition according to any one of the preceding claims, characterized in that the branched sulphonic polyester is a polymer formed by polycondensation
(a) of at least one dicarboxylic acid not bearing a sulphonic function,
(b) of at least one diol or a mixture of a diol and a diamine,
(c) of at least one monomer comprising two reactive functions, which may be identical or different, chosen from hydroxyl, amino and carboxyl groups, and also bearing at least one sulphonic function, and
(d) of at least one monomer comprising at least three reactive functions, which may be identical or different, chosen from hydroxyl, amino and carboxyl groups.

5. Styling composition according to claim 4, characterized in that the branched sulphonic polyester also contains units derived from difunctional monomers (e) chosen from hydroxy carboxylic acids and amino carboxylic acids, or a mixture thereof.

6. Styling composition according to claim 5, characterized in that the difunctional monomer (e) represents up to 40 mol% of the total amount of monomers.

7. Styling composition according to one of claims 4 to 6, characterized in that the monomer (c) bearing at least one sulphonic function represents from 2 mol % to 15 mol % of the total amount of monomers.

8. Styling composition according to any one of claims 4 to 7, characterized in that the monomer (d) bearing at least three reactive functions represents from 0.1 mol % to 40 mol % of the total amount of monomers.

9. Styling composition according to any one of claims 4 to 8, characterized in that the dicarboxylic acids not bearing a sulphonic function forming the units (a) are chosen from aliphatic dicarboxylic acids, alicyclic dicarboxylic acids and aromatic dicarboxylic acids, and mixtures of such acids.

10. Styling composition according to claim 9, characterized in that the dicarboxylic acids forming the units (a) are chosen from the group formed by 1,4-cyclohexanedioic acid, succinic acid, glutaric acid, adipic acid, azelaic acid, sebacic acid, fumaric acid, maleic acid, 1,3-cyclohexanedioic acid, phthalic acid, terephthalic acid and isophthalic acid, and a mixture of such acids.

11. Styling composition according to any one of claims 4 to 10, characterized in that the diols forming the units (b) are chosen from alkanediols and polyalkylenediols.

12. Styling composition according to claim 11, characterized in that the diols forming the units (b) are chosen from ethylene glycol, propylene glycol, diethylene glycol, triethylene glycol and polypropylene glycol.

13. Styling composition according to any one of claims 4 to 12, characterized in that the diamines forming the units (b) are chosen from alkanediamines and poly(oxyalkylene)diamines.

14. Styling composition according to any one of claims 4 to 13, characterized in that the monomer (c) bearing at least one sulphonic function is chosen from dicarboxylic acids, dicarboxylic acid esters, glycols and hydroxy acids, all bearing at least one sulphonic group, in acid and/or neutralized form, preferably in neutralized form.

15. Styling composition according to any one of claims 4 to 14, characterized in that the multifunctional monomer (d) is chosen from trimethylolethane, trimethylolpropane, glycerol, pentaerythritol, sorbitol, trimellitic anhydride, erythritol, threitol, dipentaerythritol, pyromellitic dianhydride and dimethylpropionic acid.

16. Styling composition according to any one of the preceding claims, characterized in that the branched sulphonic polyester represents from 0.2% to 15% and preferably from 0.5% to 10% of the total weight of the liquid phase of the styling composition.

17. Styling composition according to any one of the preceding claims, characterized in that the water content of the liquid phase is between 70% and 98% and preferably between 75% and 96% by weight.

18. Styling composition according to any one of the preceding claims, characterized in that it is a hair lacquer.

19. Process for styling by spraying a styling composition, packaged in an aerosol device, according to any one of the preceding claims, onto the hair.
